(19) United States
(12) Patent Application Publication
(10) Pub. No.: US 2003/0087285 A1
(43) Pub. Date: May 8, 2003

(54) Algorithm for estimating the outcome of inflammation following injury or infection

(76) Inventors: Carson C. Chow, Pittsburgh, PA (US); Yoram Vodovotz, Sewickley, PA (US); Gilles Clermont, Wexford, PA (US)

Correspondence Address: Barbara E. Johnson, WEBB ZIESENHEIM LOGSDON ORKIN & HANSON, P.C., 700 Koppers Building, 436 Seventh Avenue, Pittsburgh, PA, 15219-1818, US

(21) Appl. No.: 10/233,166

(22) Filed: Aug. 30, 2002

Related U.S. Application Data
(60) Provisional application No. 60/316,181, filed on Aug. 30, 2001.
(60) Provisional application No. 60/318,772, filed on Sep. 12, 2001.

(51) Int. Cl. C12Q 1/68; G01N 33/554; G01N 33/569; G06F 19/00; G01N 33/48; G01N 33/50

(52) U.S. Cl. 435/6; 435/7.32; 702/19; 702/20

(57) ABSTRACT

A mathematical prognostic in which changes in a number of physiologically significant factors are measured and interpolated to determine a “damage function” incident to bacterial infection or other serious inflammation. By measuring a large number of physiologically significant factors including, but not limited to, Interleukin 6 (IL6), Interleukin 10 (IL10), Nitric Oxide (NO), and others, it is possible to predict life versus death by the damage function, dD/dt, which measures and interpolates differential data for a plurality of factors. In mammals, an IL6/NO ratio <8 at 12 hours post infection is highly predictive (60%) of mortality; also in mammals, an IL6/NO ratio <4 at 24 hours post infection is highly predictive (52%) of mortality; and an IL6/IL10 ratio in mammals of <7.5 at 24 hours post infection is highly predictive (68%) of mortality.

### CROSS REFERENCE TO RELATED APPLICATIONS

This application claims the benefit of U.S. Provisional Application Serial No. 60/316,181, filed Aug. 30, 2001, and U.S. Provisional Application Serial No. 60/318,772, filed Sep. 12, 2001, which are incorporated by reference in their entirety.

### BACKGROUND OF THE INVENTION

1. Field of the Invention

The present invention relates to a dynamic system of differential equations involving key components and interactions of the acute inflammatory response for interpretation of the inflammatory response to predict appropriate patient therapy, applicable drugs for patient therapy, and the proper timing for drug delivery.

2. Description of Related Art

Recent advances in the understanding of the systemic inflammatory response syndrome (SIRS), which is also known as sepsis, and multi-system organ dysfunction syndrome (MODS) have resulted through identification of individual components of the complicated signaling pathways and structures of the immune system by genetic and biochemical means. Systemic inflammatory response syndrome (SIRS) results from a number of symptoms manifested by patients that have sustained major systematic insults, such as trauma and infection. SIRS is outwardly characterized by a combination of fever, tachycardia, tachypnea, and hypotension. MODS may originate from a poorly controlled inflammatory response resulting in cellular dysfunction, which results in macroscopic organ system dysfunction. However, the sequence of events leading to a state of persistent inflammatory response remains unclear even though much is known about the inflammatory response.

The inflammatory response results from the dynamic interaction of numerous components of the immune system in an attempt to restore homeostasis. The homeostatic balance can be upset primarily by direct tissue injury, such as mechanical trauma, pancreatitis, tissue hypoxia, and antigenic challenge resulting from infection. In restoring homeostasis caused by infection, the immune response involves several components, which include bacteria, bacterial pro-inflammatory substances, effector cells (macrophages and neutrophils), and effector cell-derived pro- and anti-inflammatory substances. Each component plays a unique role in the immune response to infection.

Bacteria and other antigens stimulate the inflammatory response, directly or indirectly, by secreting certain products, or by the bacteria's own destruction and subsequent liberation of pro-inflammatory substances such as endotoxins. The arrival of bacteria is detected by a limited number of receptors on effector cells, which are the primary mediators of the inflammatory response.

Effector cells include neutrophils, monocytes, fixed tissue macrophages, lymphocytes, and vascular endothelial cells. Effector cell products play an integral role in the immune response and include reactive oxygen, nitrogen metabolites, eicosanoids, cytokines, and chemokines acting in an autocrine, paracrine, or endocrine fashion. Specifically, macrophages are multifunctional effector cells that play a central role in the acute inflammatory response. Macrophages are present a priori as sentinels in virtually all body tissues and, therefore, are chronologically the first responders to body insult or invasion. As a cellular population, macrophages are known to remain in a persistent state of activation while multi-system organ failure is developing. In the state of activation, macrophages secrete high levels of products such as cytokines, free radicals, and degradative enzymes. In addition to macrophages, neutrophils have an important role in the inflammatory response. Neutrophils are the most common leukocyte and are attracted to sites of injury and infection. Neutrophils are activated by bacterial products, such as peptides containing formylated methionine residues.

Bacteria and tissue injury also activate the complement pathway, causing the liberation of powerful neutrophil chemo-attractants such as C3a and C5a. These activated complement pathway molecules, in turn, activate neutrophils causing increased adhesiveness, tissue migration, degranulation, and phagocytosis of bacteria. Naive neutrophils reach compromised tissue by detecting specific surface signals on vascular endothelium and navigate to their complement and subsequent activation of neutrophils. The activated complement pathway molecules also activate macrophages.

Cytokines have a signaling role, primarily between cells of the immune system and endothelial cells. Cytokines are peptide hormones with a vast array of effects on growth, development, immunity, and diseases that are regulated in complex ways at the transcriptional, post-transcriptional, translational, and post-translational levels. A variety of cellular products that are essential to a successful immune response to the stress are expressed as a result of the direct action of cytokines. The systemic action of cytokines as part of an activated immune system internally drives the systemic inflammatory response syndrome.

Often overlapping in their spectra of action, cytokine activities include interaction with one another, and regulation of each other's expression and activity. Pro-inflammatory cytokines, such as Tissue Necrosis Factor (TNF)-α, Interleukin (IL)-1, and Interleukin (IL)-6, are involved in various stages of the inflammatory response to microbial pathogens and their secreted products. Pro-inflammatory cytokines are made by and regulate the activity of macrophages and neutrophils. Anti-inflammatory cytokines are the counterbalancing force to pro-inflammatory cytokines and include Interleukin (IL)-10 and Tissue Growth Factor (TGF)-β1. Anti-inflammatory cytokines serve to dampen the inflammatory response and hence the return to homeostasis. However, anti-inflammatory cytokines can lead to suppression of the immune system when dysregulated.

Free radicals and degradative enzymes are another component of the immune response and are produced by macrophages and neutrophils. Free radicals such as superoxide, hydroxyl radical, and hydrogen peroxide, which are known collectively as reactive oxygen species, are directly toxic to pathogens and host cells. These molecules also serve a signaling role by inducing the production of pro-inflammatory cytokines. The free radical nitric oxide and the products derived from its reaction with numerous molecules including reactive oxygen species are known collectively as reactive nitrogen species. (The blood ionic form of reactive Nitrogen Species is Nitrate NO₃- and Nitrite NO₂-.) These molecules can be cytotoxic or cytostatic to pathogens, and may help protect host cells from damage. However, the elevated levels of nitric oxide produced systemically upon infection can have adverse hemodynamic effects. In addition, degradative enzymes found in the granules of both neutrophils and macrophages serve to break down engulfed bacteria, and indirectly serve a signaling role by causing the release of bacterial products that, in turn, are pro-inflammatory.

Advances in understanding of the mediators of the inflammatory response have led to mechanistic rationales for the development of targeted treatments in sepsis and other diseases characterized by uncontrolled inflammation. Currently, several molecular targets are being investigated for the treatment of destructive inflammation. The therapeutic agents under investigation are anti-cytokine antibodies, soluble cytokine receptors, cyclooxygenase inhibitors, neutrophil-endothelial adhesion blockers, and nitric acid donor or scavenger molecules. Despite promising results in animal and human trials, large-scale trials of therapies targeted at inhibiting or scavenging various inflammatory mediators at the global inflammatory response have generally failed to improve survival. Although many reasons such as the wrong rationale, questionable drug activity, faulty patient selection, and insensitive end-points, may explain the failure of the trials, the most likely explanation is that acute inflammation represents the highly integrated response of a complex adaptive immune system. Targeting one sub-mechanism of the inflammatory response will result, at best, in a modest modulation of the integrated inflammatory response.

The complexity of the molecular and genetic pathways involved in the acute response to injury has resulted in confining experimentation to the isolated aspects of the innate immune response, and intimidation about gaining an integrated description of the acute inflammatory response. Although there have been advances in understanding the complex molecular physiology of the acute inflammatory response, the reasons underlying the immune system pathways and the association between molecular events and organ dysfunction remain elusive. There has been no published attempt to model the acute inflammatory response quantitatively, presumably because of the perceived untenable complexity of the physiological response. Mathematical models that include several possible mechanisms relating inflammatory effectors and end organ damage could provide a means to correlate time-dependent patterns of effectors with outcome.

The inflammatory response to bacterial infection can be modeled by using a system of differential equations that expresses the time variations of individual components simultaneously. Such a dynamic systems approach can provide an intuitive means to translate mechanistic concepts into a mathematical framework, be analyzed using a large body of existing techniques, be numerically simulated easily and inexpensively on a desktop computer, provide both qualitative and quantitative predictions, and allow the systematic incorporation of higher levels of complexity. Therefore, there is a present need for a simplified system of mathematical equations that involves key components and interactions of the acute inflammatory response to predict which patients are to be treated, the drugs to use to treat those patients, and the proper timing for delivery of the drugs.

### SUMMARY OF THE INVENTION

In order to meet this need, the present invention is a mathematical prognostic and model in which changes in a number of physiologically significant factors are measured and interpolated to determine a “damage function” incident to bacterial infection or other serious inflammation. By measuring a large number of physiologically significant factors including, but not limited, to Interleukin 6 (IL6), Interleukin 10 (IL10), Nitric Oxide (NO), and others, it is possible to predict life versus death by the damage function, dD/dt, which measures and interpolates differential data for a plurality of factors. Certain ratios of these physiologically significant factors, measured at given points in time, are representative of the damage function without embodying the damage function in its entirety, but the ratios are useful nonetheless. For example, in mammals an IL6/NO ratio <8 at 12 hours post infection is highly predictive (60%) of mortality; also in mammals an IL6/NO ratio <4 at 24 hours post infection is highly predictive (52%) of mortality; and an IL6/IL10 ratio in mammals of <7.5 at 24 hours post infection is highly predictive (68%) of mortality. Either by determination of the damage function in entirety, or by observation of the IL6/NO and/or IL6/IL10 levels at appointed times, prognosis of patient outcome is possible which prognosis, in turn, suggests appropriate intervention. As a model for active agent analysis, the mathematical model and the damage function, in particular, may be used to create simulated clinical trials, in which real patient data from bacterial infection situations is analyzed and analogized to animal model studies of active agents in order to amplify the significance of the animal model results.

### BRIEF DESCRIPTION OF THE DRAWINGS

FIG. 1 shows several graphs illustrating the time dependent behavior of the system;

FIG. 2 shows several graphs illustrating a deficient neutrophil as being quite deficient in producing pro-inflammatory cytokines;

FIG. 3 shows graphs illustrating a high baseline concentration of anti-inflammatory mediators leading to reduced expression of pro-inflammatory substances and effectors;

FIG. 4a shows several graphs illustrating the effect of pathogen inoculum size on pathogen multiplication;

FIG. 4b shows several graphs illustrating pathogen growth effect;

FIG. 4c shows a graph illustrating bifurcation, which is the irreversible impact on blood pressure caused by pathogen growth rate;

FIG. 5 shows several graphs illustrating the possibility of therapeutic intervention simulating the administration of an antibiotic through the convergence of several parameters of the system in a complicated, but suggestive, manner for a quantitative evaluation of the impact of therapeutic strategies; and

FIG. 6 shows a graph illustrating the use of the system to predict the effects of administration of a substance that “soaks” the nominal endotoxin.

### DESCRIPTION OF THE PREFERRED EMBODIMENTS

As described above, the present invention is a mathematical model in which changes in a number of physiologically significant factors are measured and interpolated to determine a “damage function” incident to bacterial infection or other serious inflammation. By measuring a large number of physiologically significant factors including, but not limited to, Interleukin 6 (IL6), Interleukin 10 (IL10), Nitric Oxide (NO), and others, it is possible to predict life versus death by the damage function, dD/dt, which measures and interpolates differential data for a plurality of factors. Certain ratios of these physiologically significant factors, measured at given points in time, are representative of the damage function without embodying the damage function in its entirety, but the ratios are useful nonetheless. For example, in mammals an IL6/NO ratio <8 at 12 hours post infection is highly predictive (60%) of mortality; also in mammals an IL6/NO ratio <4 at 24 hours post infection is highly predictive (52%) of mortality; and an IL6/IL10 ratio in mammals of <7.5 at 24 hours post infection is highly predictive (68%) of mortality. Either by determination of the damage function in entirety, or by observation of the IL6/NO and/or IL6/IL10 levels at appointed times, prognosis of patient outcome is possible which prognosis, in turn, suggests appropriate intervention. As a model for active agent analysis, the mathematical model and the damage function, in particular, may be used to create simulated clinical trials, in which real patient data from bacterial infection situations is analyzed and analogized to animal model studies of active agents in order to amplify the significance of the animal model results.

Stated another way, the present invention is a simplified system of differential equations that incorporates key components and interactions of the acute inflammatory response to predict which patients are to be treated, the drugs to use to treat those patients, and the proper timing for delivery of the drugs. The system is capable of specific and clinical predictions for treating the early response to external biological challenges while taking into account several of the main effector mechanisms currently known. The system can be used to predict the outcome of common clinical interventions performed as part of the management of patients with SIRS as well as reanalyzing the data from previously published studies on sepsis. The system includes variables that recognize the possibility of clinical interventions, such as antibiotics or other molecular therapies. In addition, the system includes variables that recognize the generation of antibiotic resistance, which is a major clinical problem in the management of SIRS.

Systems software can be designed to implement the system to assist clinicians in the management of patients with SIRS. The designed software could implement a standard program capable of being run on a computer, such as a web-based program, in the form of a bedside workstation device, or as a wireless handheld device to be used by the treatment team. The devices could interface with the hospital's patient database to provide real-time diagnostic data for processing by the system to suggest courses of treatment. The system could also be applied in distance consulting, wherein data could be collected from a patient from a remote location and inputted into the software implementing the system, so that a consulting physician could suggest therapies for a specific patient.

An automated patient management system would act on diagnostic data input to deliver the appropriate treatment to a septic patient. This system would have self-correcting capabilities, adjusting the timing and dosage of interventions as the patient's condition changes. Such a system could act to stabilize a patient prior to standard hospital care. Such a system might be envisioned to be of use in military applications and remote locations as well as to paramedic personnel in civilian settings. In addition, the automated patient system could be used for offering consulting services.

The current management of a patient suffering from acute injury or infection is largely resuscitative and supportive of organ function, such as mechanical ventilation, vasopressor medications, dialysis, etc. Active interventions consist of antibiotic administration and surgery, which are performed based on limited data and understanding and are often administered without sufficient understanding of the dynamic processes that are occurring in a patient.

The system in the present invention, if translated to any of the possible devices described, would enable clinicians to intervene much more effectively in order to treat a patient with SIRS. Currently, clinical trials testing candidate drugs for treatment of the underlying inflammatory response caused by SIRS have failed to prove effective. The trials have failed to take into consideration the dynamic nature of SIRS in an individual patient, and have not been set up to address fluctuations the parameters accounted for in the present invention. Clinical trials would benefit from a rational prediction of the type and timing of interventions to perform in an individual patient. Therefore, the present invention would improve the state-of-the-art in design and implementation of clinical trials by allowing individualization of treatment. At a minimum, the present invention would rule out types of interventions that are unlikely to succeed, and identify viable therapies that would maximize efficacy of treatment.

The system includes time variations of individual components simultaneously. This approach provides an intuitive means to translate mechanistic concepts of the inflammatory response into a mathematical framework. The inflammatory response can be analyzed using a large body of existing techniques that can be numerically simulated easily and inexpensively on a desktop computer. The inflammatory response provides qualitative and quantitative predictions and allows for the systematic incorporation of higher levels of complexity. The system also gives consideration to the characteristics of pathogens and the host because a considerable amount of information is available on the kinetics of individual pathogens and antibiotic responsiveness. These variables are contained in the equations of the system that can be optimized for each individual during an initial observation phase.

Generally, the system is comprised of multiple differential equations, which describe the interaction between initiator, effector, and target components of the early inflammatory response. In combination, the differential equations constitute an algorithm to predict a patient's local and systemic response to a localized infection. The variables in the equations are described in Table 1. The interaction between the different components of the dynamical system is based on a principal of mass-action kinetics.

In the first embodiment, the system is comprised of the following 11 differential equations:

$$\begin{matrix}\frac{ⅆ p}{ⅆ t} = {k}_{p1} p ( 1 - {k}_{p2} {p}^{2} ) - ( {k}_{pm} {f}_{2} ( {m}_{a} , {T}_{m a} ) + {k}_{p n e} f ( {n}_{e} , {T}_{n e} ) ) p - {k}_{p A} A p + P ( t ) & ( 1 ) \\ \frac{{Dp}_{c}}{ⅆ t} = {k}_{pc1} p ( {k}_{pm} f ( {m}_{a} , {T}_{a} ) + {k}_{p n e} f ( {n}_{e} , {T}_{n e} ) ) + {k}_{pc2} p - {k}_{pc3} {p}_{c} + C ( t ) & ( 2 ) \\ \frac{ⅆ {p}_{e}}{ⅆ t} = {k}_{pe1} p ( {k}_{pm} f ( {m}_{a} , {T}_{a} ) + {k}_{p n e} f ( {n}_{e} , {T}_{n e} ) ) + {k}_{pe2} p - {k}_{pe3} {p}_{e} & ( 3 ) \\ \frac{ⅆ {m}_{a}}{ⅆ t} = {m}_{a} ( 1 - {k}_{ma3} {m}_{a}^{2} ) ( {k}_{m1c} f ( p + {p}_{c} , {T}_{p} ) + {k}_{m1e} f ( {p}_{e} , {T}_{p e} ) + {k}_{m n p} f ( {n}_{p} , {T}_{n p} ) ) - {k}_{ma2} {m}_{a} + {C}_{m} & ( 4 ) \\ \frac{ⅆ n}{ⅆ t} = n ( 1 - {k}_{n4} {n}^{2} ) ( {k}_{n1c} f ( p + {p}_{c} , {T}_{p} ) + {k}_{n1e} f ( {p}_{e} , {T}_{p e} ) + n ( - {k}_{n2} {n}_{e} - {k}_{n3} ) + {C}_{n} & ( 5 ) \\ \frac{ⅆ {n}_{e}}{ⅆ t} = ( 1 - f ( {n}_{a} , {T}_{n a} ) / {T}_{n a} ) ( {k}_{ne1} n + {k}_{ne2} {m}_{a} ) f ( {n}_{p} , {T}_{p} ) - {k}_{ne3} {n}_{e} & ( 6 ) \\ \frac{ⅆ {n}_{p}}{ⅆ t} = ( 1 - f ( {n}_{a} , {T}_{n a} ) / {T}_{n a} ) ( {k}_{np1} n + {k}_{np2} {m}_{a} ) f ( {n}_{p} , {T}_{p} ) - {k}_{np3} {n}_{p} & ( 7 ) \\ \frac{ⅆ {n}_{a}}{ⅆ t} = {n}_{a r} - {k}_{n a 4} {n}_{a} & ( 8 ) \\ \frac{ⅆ {n}_{a r}}{ⅆ t} = - {k}_{na1} {n}_{ar} + {k}_{na2} f ( n , {T}_{n} ) - {k}_{na3} f ( {m}_{a} , {T}_{m a} ) & ( 9 ) \\ \frac{ⅆ B}{ⅆ t} = - ( {k}_{bp1} {p}_{e} + {k}_{bp2} {n}_{p} ) B + {B}_{0} - B & ( 10 ) \\ \frac{ⅆ A}{ⅆ t} = - a {k}_{p1} A + S ( t ) & ( 11 )\end{matrix}$$

Equation 1 describes the population behavior of pathogens. A bacterial pathogen P is externally introduced within the time course C(t) and multiplies exponentially. The system conceptually includes the property of macrophages mₐ as well as neutrophils n and reactive oxygen and nitrogen species nₑ, which is a killing substance released by both macrophages mₐ and neutrophils n.

Equation 2 describes the different mechanisms by which pathogens cause inflammation. The pathogens promote inflammation through a complement-like substance p_{c} and an endotoxin-like substance pₑ. Pathogens coated with a complement-like substance p_{c} attract the effector cells and stimulation the activation of the stimulator cells.

Equation 3 describes the sequence of interactions surrounding the liberation and localized spread of endotoxin pₑ induced by bacterial pathogens. Although endotoxins pₑ accompanies live pathogens, destruction of pathogens by macrophages mₐ, neutrophils n, and eventually antibiotic agents is related to temporary increase in the liberation of endotoxins pₑ. The intiator pₑ does not multiply, but undergoes catabolism and can efflux from the site of infection and cause inflammation in target organs. This sequence of interactions is also detailed in the relevant term of Equation 10. Although bacterial invasion is the leading paradigm of this simplified model, the inclusion of several constants in the model allows the simulation of a variety of pathogens. For example, direct tissue damage, such as trauma, would not generate intact pathogens p, but rather a complement-like effector substance p_{c} according to a time dependent function C(t).

The cellular effector components included in the model are macrophages mₐ and neutrophils n. Five types of soluble effectors are also included in the model. More neutrophils n and macrophages mₐ will be activated secondarily to the presence of intact pathogens, inert soluble pathogenic components such as a complement-like substances p_{c} or endotoxins pₑ, or a soluble pro-inflammatory effector substance nₚ. Activated macrophages can die at a baseline rate or be deactivated by the presence of anti-inflammatory effector substance nₐ. The macrophage dynamic is detailed in Equation 4. Neutrophils are governed by a similar dynamic, except that the rates of activation and deactivation are higher than for macrophages. In addition, it is assumed that endotoxin-like substance pₑ could activate neutrophils directly. The model allows the flexibility to separate the ability of the neutrophil to produce pro-inflammatory effector substance nₚ and the ability to release reactive oxygen and nitrogen species nₑ, because each are clearly stimulated and inhibited by different processes. This is conveyed by the use of different rates of production of these products in Equation 6 and Equation 7. The neutrophil dynamic is detailed in Equation 5. The reactive oxygen and nitrogen species nₑ are produced by both macrophages mₐ and neutrophils n, but their ability to produce these effector molecules is saturable and modulated by the presence of soluble anti-inflammatory effector substances nₐ. This dynamic is detailed in Equation 6.

The generation of a soluble pro-inflammatory effector substance nₚ follows a similar dynamic, with different rates. The soluble anti-inflammatory substances nₐ are produced by both macrophages mₐ and neutrophils n, but their appearance is delayed with respect to pro-inflammatory effector substances. In this system, the rate of production of soluble anti-inflammatory effector substances nₐ is linked to the effector cells, not the concentration of soluble pro-inflammatory effector substances nₚ. On the other hand, the action of both soluble pro-inflammatory effector substance nₚ and soluble anti-inflammatory effector substances nₐ either shorten or prolong cell life, which reflects their respective contribution on the timing of apoptotic cell death. This dynamic is described in Equation 7, Equation 8, and Equation 9.

In the system, the model target tissue is a generic arteriole without attempting to separate smooth muscle cells and endothelium. The principle used is that the arteriole is responsible for generating the observed physiologic variable of vascular tone (as a proxy to systemic blood pressure). Vascular tone is influenced directly by effector components effluxing from the primary site of inflammation, but only once the concentration of effector agent at the primary site exceeds a predetermined threshold. It is hypothesized that soluble effectors such as endotoxins pₑ and soluble pro-inflammatory effector substances nₚ effluxed at lower concentrations than effector cells. We also assumed that soluble effectors such as endotoxins pₑ were more potent than soluble pro-inflammatory effector substances nₚ in generating a hypotensive response. This dynamic is described in Equation 10.

Finally, Equation 11 describes the dynamic of an extrinsic intervention that results in pathogen killing.

Table 1 describes the components of the acute inflammatory response as used in the first embodiment of the system.

TABLE 1
Components of the Acute Inflammatory Response
included in the System

| COMPONENTS | DESCRIPTION | EXAMPLES |
|---|---|---|
| Initiator | | |
| p | Intact pathogen, can multiply | Bacteria |
| p_{c} | Inert pathogenic component that | Complement |
| | can attract and activate effector | |
| | cells | |
| pₑ | Inert pathogenic component that | Endotoxin |
| | activates effector cells and be | |
| | transported to distant sites | |
| Effector | | |
| mₐ | First effector cell to be | Macrophage |
| | activated, acts as general activator, | |
| | produces some soluble effectors | |
| n | Second effector cell, produces | Neutrophils |
| | soluble effectors that destroys p | |
| nₑ | Soluble effector produced by n | Reactive oxygen |
| | and m, kills intact pathogens | and nitrogen |
| | | species |
| | | degradative |
| | | enzymes |
| nₚ | Soluble “pro-inflammatory” | TNF-α, IL6 |
| | effector | |
| nₐ | Soluble “anti-inflammatory” | IL10, TGF-β₁ |
| | effector | |
| nₐᵣ | Anti-inflammatory delay variable, | |
| | as these are generally expressed | |
| | later than pro-inflammatory | |
| | effectors | |
| Target | | |
| B | A physiologic observable, such as | Blood pressure |
| | blood pressure, that correlates | |
| | with global outcome | |
| Intervention | | |
| A | An extrinsic modulator of the | Antibiotic |
| | response which enhances the | |
| | killing of pathogen | |

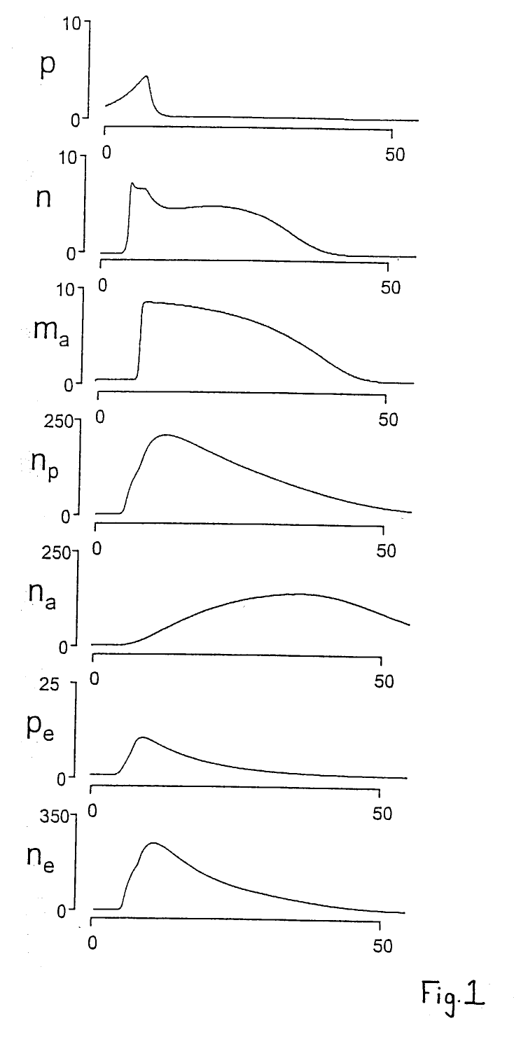

In initial experiments with the system, variables were run while considering localized processed concentration of various variables included in the model, and the effect of spill-out of effectors on blood pressure. The purpose of the initial runs was to obtain a description of events in several scenarios, reflecting common clinical situations. As shown in FIG. 1, the time dependent behavior of the system is shown, wherein the concentrations (y-axis) and time (x-axis) are not calibrated. The usefulness of this data is limited to the qualitative behavior of the system.

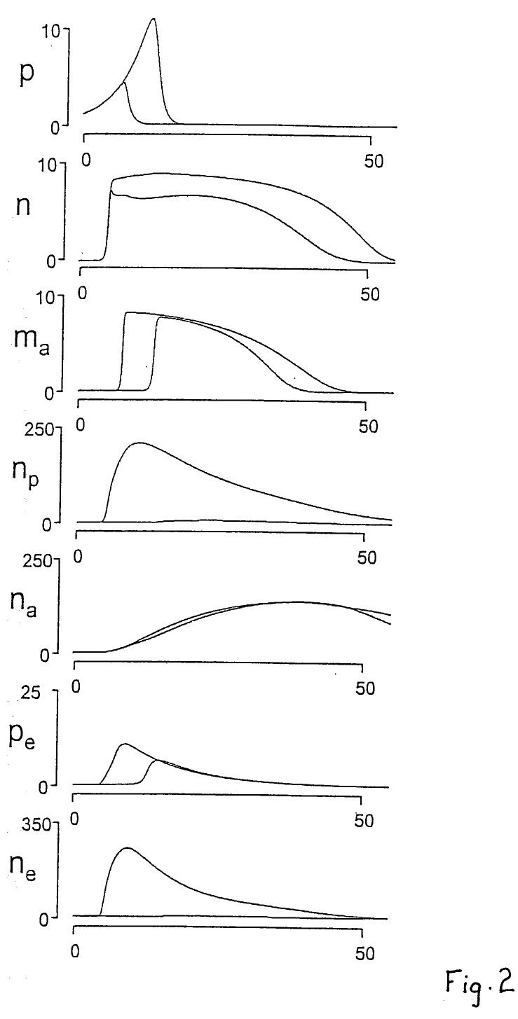

As shown in FIG. 2, a deficient neutrophil is quite deficient in producing pro-inflammatory cytokines. Pathogens typically grow to a larger population, but are nevertheless cleared by the combined action of macrophages and their effectors. However, if the system simulation is allowed to run for longer time periods, pathogens reappear.

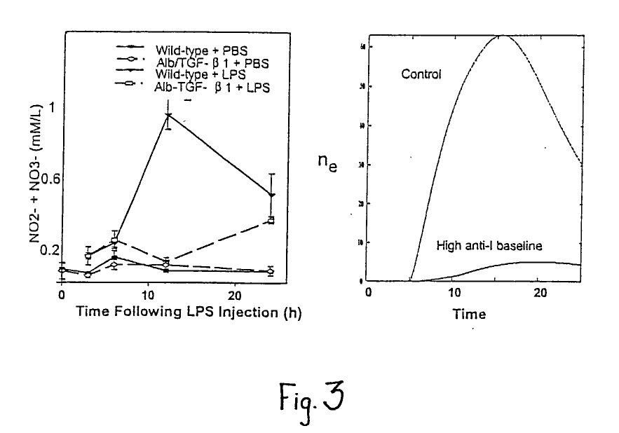

As shown in FIG. 3, a high baseline concentration of anti-inflammatory mediators leads to reduced expression of pro-inflammatory substances and effectors, such as nitric oxide. In this experiment, the over expression of TGF-β1 in mice had significantly reduced production of NO related substances (serum nitrites and nitrates) when administered lipopolysaccharide (LPS) when compared to wild-type mice or mice administered placebo (PBS).

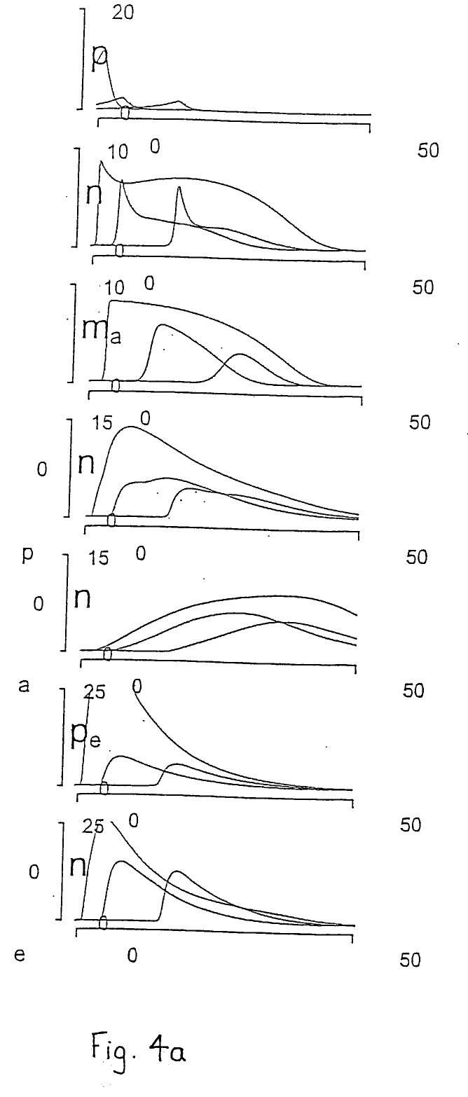

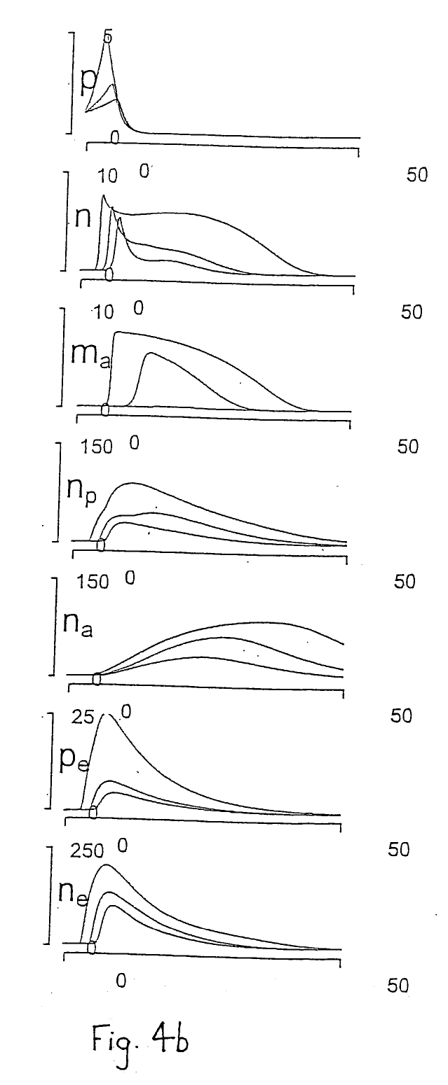

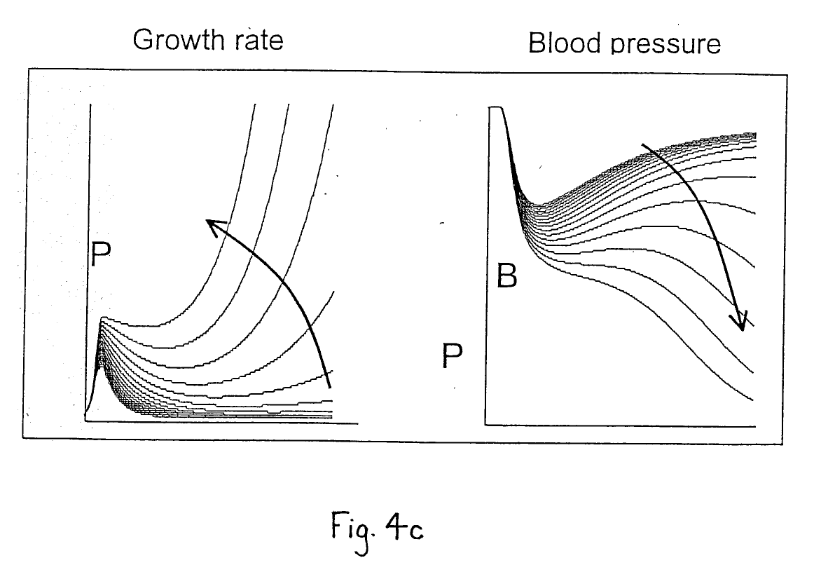

FIGS. 4a-4c show the multiplication rates of pathogens and how different sizes of pathogen inocula affect pathogen growth rates. As illustrated in FIGS. 4a-4c, the growth rate of the pathogen is clearly more important than the size of the inoculum. This information is important because the system can predict a threshold growth rate at which the immune defense mechanisms are incompetent to control the infection. The system can monitor pathogen growth and link that data with a catastrophic drop in blood pressure to show the death of a patient.

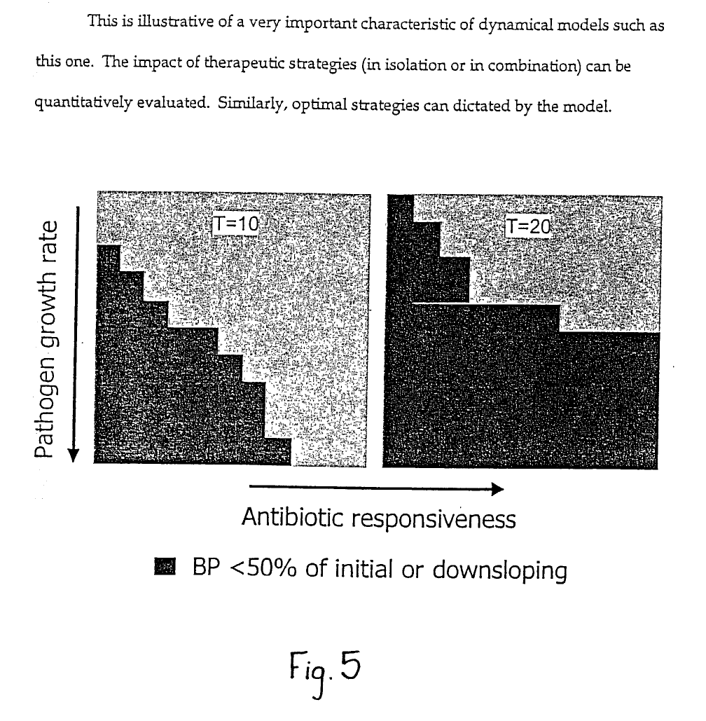

As shown in FIG. 5, a therapeutic intervention simulating the administration of an antibiotic can be used to predict the effect of a antibiotic on a patient. A substance that directly killed pathogens was introduced with a user-specific efficacy. The efficacy was decreased over time to simulate the gradual loss of efficacy of antibiotics as resistant pathogens are selected. As expected, administration of antibiotics assists in the more rapid control of an infection. An effective antibiotic will help control an infection that would otherwise be lethal. However, later intervention with an antibiotic, prior to death, will result in considerably less impact of an otherwise effective antibiotic on death. The convergence of several parameters of the system in a complicated manner can be accomplished by the system. Increased antibiotic effectiveness results in better eradication of pathogens and presumably better survival. Increased growth rate of pathogen results in worse survival. Earlier administration of antibiotic may save lives, everything else being equal. “Death” means a decrease by more than 50% of blood pressure or down-sloping of blood pressure at the end of the simulation (t=50). The simulation provides a prediction of the outcome (in blood pressure) given bacterial growth rate and antibiotic efficacy and the quantitative evaluation of the impact of therapeutic strategies in isolation or in combination.

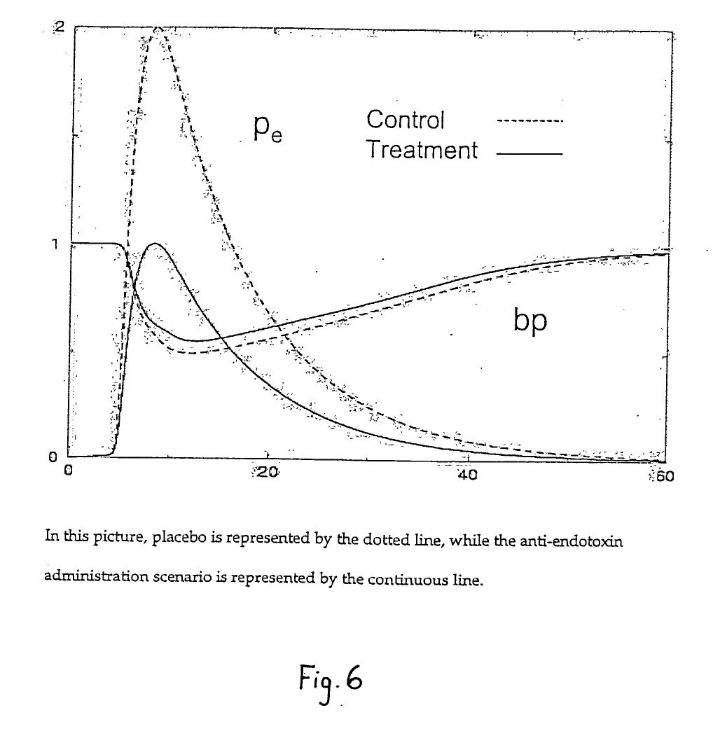

As shown in FIG. 6, the system can be used to predict the effects of administering a “soaking” substance, such as endotoxin pₑ FIG. 6 shows that the final effect on blood pressure is marginal, even though more than 50% by surface area if the endotoxin was soaked. The marginal effect on blood pressure occurs because more than one factor in the model is responsible for the decrease in blood pressure. Quantifying the relative importance of different processes to impact outcome is of paramount importance in the design of medical therapies. If endotoxin was the major factor contributing to lower the blood pressure, the results obtained from the system would show a major impact from an anti-endotoxin therapy.

In the second embodiment, the system includes a more detailed model of acute inflammation variables. The following 16 differential equations comprise the second embodiment of the system:

$$\begin{matrix}\frac{D P}{D t} = {k}_{p} P ( 1 - {k}_{P s} P ) - ( {k}_{P M} {M}_{a} + {k}_{P O2} {O}_{2} + {k}_{P N O} N O + A B ( t ) ) P + {S}_{P} ( t ) & ( {1}^{′} ) \\ \frac{D P E}{D t} = ( {k}_{P} {M}_{a} + {k}_{P O2} {O}_{2} + {k}_{P N O} N O + A B ( t ) P - {k}_{P E} P E + {S}_{P E} ( t ) & ( {2}^{′} ) \\ \frac{D {M}_{r}}{D t} = - ( {k}_{M P} p + {k}_{M P E} P E + {k}_{MD} D ) ( f ( {C}_{p} ) + f ( I L6 ) ) {f}_{s} ( {C}_{a} ) + {k}_{Mg} f ( {M}_{a} + {C}_{p} + N O + P E ) - {k}_{M} {M}_{r} & ( {3}^{′} ) \\ \frac{D {M}_{a}}{D t} = ( {k}_{M P} p + {k}_{pe} P E + {k}_{md} D ) ( f ( {C}_{p} ) + f ( I L6 ) ) {f}_{s} ( {C}_{a} ) - {k}_{ma} {M}_{a} & ( {4}^{′} ) \\ \frac{D N}{D t} = ( {k}_{NP} P + {k}_{NPE} P E + {k}_{NCP} {C}_{P} + {k}_{NIL6} I L6 + {k}_{ND} D ) N - ( {k}_{NNO} N O + {k}_{NO2} O2 ) N - {k}_{N} {f}_{s} ( {C}_{p} ) N & ( {5}^{′} ) \\ \frac{D {O}_{2}}{D t} = ( ( {k}_{O2N} N + {k}_{O2M} {M}_{a} ) ( f ( {C}_{p} ) + f ( I L6 ) ) + {k}_{O2NP} N P ) {f}_{s} ( {C}_{a} ) - {k}_{O2} {O}_{2} & ( {6}^{′} ) \\ \frac{D N}{\frac{O}{D t}} = ( {k}_{NON} N + {k}_{NOM} {M}_{a} ) ( f ( {C}_{p} ) + f ( I L6 ) ) {f}_{s} ( {C}_{a} ) - {k}_{NO} N O & ( {7}^{′} ) \\ \frac{D {C}_{p}}{D t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{D I L}{\frac{6}{D t}} = {k}_{IL6M} {M}_{a} {f}_{s} ( {C}_{a} ) - {k}_{IL6} I L6 & ( {9}^{′} ) \\ \frac{D {C}_{a r}}{D t} = ( {k}_{CaN} N + {k}_{CaM} {M}_{a} ) f ( {C}_{p} + N O + {O}_{2} ) - {k}_{Car} {C}_{ar} & ( {10}^{′} ) \\ \frac{D {C}_{a}}{D t} = {C}_{ar} - {k}_{Ca} {C}_{a} + {S}_{PC} ( t ) & ( {11}^{′} ) \\ \frac{D T F}{D t} = ( {k}_{TFPE} P E + {k}_{TFCp} {C}_{p} + {k}_{TFIL6} I L6 ) {f}_{s} ( P C ) - {k}_{T F} T F - ktf ( t ) T F & ( {12}^{′} ) \\ \frac{D T H}{D t} = T F ( 1 + {k}_{THn} T H ) - {k}_{TH} T F \\ \frac{ⅆ T H}{ⅆ t} = T F ( 1 + {k}_{THn} T H ) - {k}_{TH} T F & ( {13}^{′} ) \\ \frac{D P C}{D t} = {k}_{PCTH} T H - {k}_{PC} PC + {S}_{PC} ( t ) & ( {14}^{′} ) \\ \frac{D B P}{D t} = {k}_{BP} ( 1 - B P ) - {k}_{{BPO}_{2}} {O}_{2} {f}_{s} ( N O ) + {k}_{{BPC}_{p}} {C}_{p} + {k}_{BPTH} T H ) B P & ( {15}^{′} ) \\ \frac{D D}{D t} = {k}_{DBP} ( 1 - B P ) + {k}_{DCp} {C}_{p} + {k}_{DO2} {O}_{2} + {k}_{DNO} N O / ( 1 + N O ) + {k}_{DEq} g ( {O}_{2} , N O ) - {k}_{D} D & ( {16}^{′} )\end{matrix}$$

The equations in the second embodiment incorporate pathogen P, endotoxin Pₑ, resting and active macrophages Mᵣ and Mₐ, respectively, neutrophils N, two effector molecules NO and O₂, a short term pro-inflammatory cytokine Cₚ, a longer term cytokine IL6, and an anti-inflammatory cytokine Cₐ. This system also includes recognition of a coagulation system represented by tissue factor TF, thrombin TH, and activated protein P_{C}. This system recognizes a blood pressure variable BP and a tissue dysfunction/damage variable D. Similar to the first embodiment, there is a source term for pathogens and endotoxins as well as an antibiotic term to eliminate pathogens. Antibiotic resistance is incorporated into the system by reducing the efficacy of pathogen elimination by antibiotics in a time-dependent way. Effective therapies, such as mechanisms for clearing pro-inflammatory cytokines, and means of enhancing the supply of anti-inflammatory cytokines and activated protein C, are included in the system. The blood pressure variable can be lowered to simulate the effects of trauma by inducing damage and hemorrhaging.

The present invention can be calibrated to capture the quantitative aspects of the object being modeled. A calibrated system is capable of estimating concentrations and the actual variations of those concentrations, or other physiologic parameters such as cell count and blood pressure, over time. The estimation of the various rates is derived from the literature, when available, or from educated guesses, and comparing the dynamic description obtained from the empirical data. The system contains approximately 50 parameters, most of which reflect the relative importance of certain processes, such as cell or effector half-lives, as well as the phenomena of biological saturation or exhaustion, where the effects of positive feedback are limited.

The system must be optimized to embrace the primary goal of the system to predict which interventions, as shown by modifications in the dynamic structure of the model, would most significantly alter a measurable outcome. For example, a decrease in blood pressure will result in death, an undesirable event in most circumstances in critically ill patients. Some parameters are static, while others can be modified within certain limits. The process of optimization involves the steps of defining the quantity to optimize, determining a selection of parameters that can be varied in the process of optimization, determining a realistic range over which any of these parameters can be varied, choosing an optimization technique, and verifying the face validity of the results of the procedure. In most circumstances of immediate concern, the initial conditions are fixed, so one is not in search of a global optimal solution, but of a local one. This is important to know, because this knowledge would dictate that interventions are futile and outcome certain, good or bad. The framework of differential equations to express non-linear dynamics is more favorable than more heuristic methods of representing the problem if optimization is a major issue. Although alternative frameworks can be created, discrete event simulation could also be used, optimizing such representations is particularly challenging.

The invention claimed is:

1. A method for prognosing the life or death outcome of an animal or patient in which bacterial infection or inflammation is present, comprising measuring at least two physiological factors significant to the progress of bacterial infection or inflammation and predicting the likelihood of death.

2. The method according to claim 1, wherein the likelihood of death is governed by a damage function dD/dt, and wherein the damage function dD/dt is determined according to the differential equations:
$$\begin{matrix}\frac{D P}{D t} = {k}_{p} P ( 1 - {k}_{P s} P ) - ( {k}_{P M} {M}_{a} + {k}_{P O2} {O}_{2} + {k}_{P N O} N O + A B ( t ) ) P + {S}_{P} ( t ) & ( {1}^{′} ) \\ \frac{D P E}{D t} = ( {k}_{P} {M}_{a} + {k}_{P O2} {O}_{2} + {k}_{P N O} N O + A B ( t ) P - {k}_{P E} P E + {S}_{P E} ( t ) & ( {2}^{′} ) \\ \frac{D {M}_{r}}{D t} = - ( {k}_{M P} p + {k}_{M P E} P E + {k}_{MD} D ) ( f ( {C}_{p} ) + f ( I L6 ) ) {f}_{s} ( {C}_{a} ) + {k}_{Mg} f ( {M}_{a} + {C}_{p} + N O + P E ) - {k}_{M} {M}_{r} & ( {3}^{′} ) \\ \frac{D {M}_{a}}{D t} = ( {k}_{M P} p + {k}_{pe} P E + {k}_{md} D ) ( f ( {C}_{p} ) + f ( I L6 ) ) {f}_{s} ( {C}_{a} ) - {k}_{ma} {M}_{a} & ( {4}^{′} ) \\ \frac{D N}{D t} = ( {k}_{NP} P + {k}_{NPE} P E + {k}_{NCP} {C}_{P} + {k}_{NIL6} I L6 + {k}_{ND} D ) N - ( {k}_{NNO} N O + {k}_{NO2} O2 ) N - {k}_{N} {f}_{s} ( {C}_{p} ) N & ( {5}^{′} ) \\ \frac{D {O}_{2}}{D t} = ( ( {k}_{O2N} N + {k}_{O2M} {M}_{a} ) ( f ( {C}_{p} ) + f ( I L6 ) ) + {k}_{O2NP} N P ) {f}_{s} ( {C}_{a} ) - {k}_{O2} {O}_{2} & ( {6}^{′} ) \\ \frac{D N}{\frac{O}{D t}} = ( {k}_{NON} N + {k}_{NOM} {M}_{a} ) ( f ( {C}_{p} ) + f ( I L6 ) ) {f}_{s} ( {C}_{a} ) - {k}_{NO} N O & ( {7}^{′} ) \\ \frac{D {C}_{p}}{D t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{D I L}{\frac{6}{D t}} = {k}_{IL6M} {M}_{a} {f}_{s} ( {C}_{a} ) - {k}_{IL6} I L6 & ( {9}^{′} ) \\ \frac{D {C}_{a r}}{D t} = ( {k}_{CaN} N + {k}_{CaM} {M}_{a} ) f ( {C}_{p} + N O + {O}_{2} ) - {k}_{Car} {C}_{ar} & ( {10}^{′} ) \\ \frac{D {C}_{a}}{D t} = {C}_{ar} - {k}_{Ca} {C}_{a} + {S}_{PC} ( t ) & ( {11}^{′} ) \\ \frac{D T F}{D t} = ( {k}_{TFPE} P E + {k}_{TFCp} {C}_{p} + {k}_{TFIL6} I L6 ) {f}_{s} ( P C ) - {k}_{T F} T F - ktf ( t ) T F & ( {12}^{′} ) \\ \frac{D T H}{D t} = T F ( 1 + {k}_{THn} T H ) - {k}_{TH} T F \\ \frac{ⅆ T H}{ⅆ t} = T F ( 1 + {k}_{THn} T H ) - {k}_{TH} T F & ( {13}^{′} ) \\ \frac{D P C}{D t} = {k}_{PCTH} T H - {k}_{PC} PC + {S}_{PC} ( t ) & ( {14}^{′} ) \\ \frac{D B P}{D t} = {k}_{BP} ( 1 - B P ) - {k}_{{BPO}_{2}} {O}_{2} {f}_{s} ( N O ) + {k}_{{BPC}_{p}} {C}_{p} + {k}_{BPTH} T H ) B P & ( {15}^{′} ) \\ \frac{D D}{D t} = {k}_{DBP} ( 1 - B P ) + {k}_{DCp} {C}_{p} + {k}_{DO2} {O}_{2} + {k}_{DNO} N O / ( 1 + N O ) + {k}_{DEq} g ( {O}_{2} , N O ) - {k}_{D} D & ( {16}^{′} )\end{matrix}$$

3. The method according to claim 2, wherein the damage function is evidenced by a value selected from the group consisting of the ratio of IL6/NO and the ratio of IL6/IL10 at a predetermined point after the onset of infection.

4. The method according to claim 3, wherein the damage function is evidenced according to the ratio of IL6/NO and further wherein when the IL6/NO ratio is <8 at 12 hours post infection, the likelihood of mortality is about 60%.

5. The method according to claim 3, wherein the damage function is evidenced according to the ratio of IL6/NO and further wherein when the IL6/NO ratio is <4 at 24 hours post infection, the likelihood of mortality is about 52%.

6. The method according to claim 3, wherein the damage function is evidenced according to the ratio of IL6/IL10 and further wherein when the IL6/IL10 ratio is <7.5 at 24 hours post infection, the likelihood of mortality is about 68%.

7. A method for evaluating a drug candidate, comprising enhancing the meaning of an animal model study by comparing inflammation or infection data from said animal study with human data collected from human clinical trials, said human data being considered according to the equations
$$\begin{matrix}\frac{D P}{D t} = {k}_{p} P ( 1 - {k}_{P s} P ) - ( {k}_{P M} {M}_{a} + {k}_{P O2} {O}_{2} + {k}_{P N O} N O + A B ( t ) ) P + {S}_{P} ( t ) & ( {1}^{′} ) \\ \frac{D P E}{D t} = ( {k}_{P} {M}_{a} + {k}_{P O2} {O}_{2} + {k}_{P N O} N O + A B ( t ) P - {k}_{P E} P E + {S}_{P E} ( t ) & ( {2}^{′} ) \\ \frac{D {M}_{r}}{D t} = - ( {k}_{M P} p + {k}_{M P E} P E + {k}_{MD} D ) ( f ( {C}_{p} ) + f ( I L6 ) ) {f}_{s} ( {C}_{a} ) + {k}_{Mg} f ( {M}_{a} + {C}_{p} + N O + P E ) - {k}_{M} {M}_{r} & ( {3}^{′} ) \\ \frac{D {M}_{a}}{D t} = ( {k}_{M P} p + {k}_{pe} P E + {k}_{md} D ) ( f ( {C}_{p} ) + f ( I L6 ) ) {f}_{s} ( {C}_{a} ) - {k}_{ma} {M}_{a} & ( {4}^{′} ) \\ \frac{D N}{D t} = ( {k}_{NP} P + {k}_{NPE} P E + {k}_{NCP} {C}_{P} + {k}_{NIL6} I L6 + {k}_{ND} D ) N - ( {k}_{NNO} N O + {k}_{NO2} O2 ) N - {k}_{N} {f}_{s} ( {C}_{p} ) N & ( {5}^{′} ) \\ \frac{D {O}_{2}}{D t} = ( ( {k}_{O2N} N + {k}_{O2M} {M}_{a} ) ( f ( {C}_{p} ) + f ( I L6 ) ) + {k}_{O2NP} N P ) {f}_{s} ( {C}_{a} ) - {k}_{O2} {O}_{2} & ( {6}^{′} ) \\ \frac{D N}{\frac{O}{D t}} = ( {k}_{NON} N + {k}_{NOM} {M}_{a} ) ( f ( {C}_{p} ) + f ( I L6 ) ) {f}_{s} ( {C}_{a} ) - {k}_{NO} N O & ( {7}^{′} ) \\ \frac{D {C}_{p}}{D t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{D I L}{\frac{6}{D t}} = {k}_{IL6M} {M}_{a} {f}_{s} ( {C}_{a} ) - {k}_{IL6} I L6 & ( {9}^{′} ) \\ \frac{D {C}_{a r}}{D t} = ( {k}_{CaN} N + {k}_{CaM} {M}_{a} ) f ( {C}_{p} + N O + {O}_{2} ) - {k}_{Car} {C}_{ar} & ( {10}^{′} ) \\ \frac{D {C}_{a}}{D t} = {C}_{ar} - {k}_{Ca} {C}_{a} + {S}_{PC} ( t ) & ( {11}^{′} ) \\ \frac{D T F}{D t} = ( {k}_{TFPE} P E + {k}_{TFCp} {C}_{p} + {k}_{TFIL6} I L6 ) {f}_{s} ( P C ) - {k}_{T F} T F - ktf ( t ) T F & ( {12}^{′} ) \\ \frac{D T H}{D t} = T F ( 1 + {k}_{THn} T H ) - {k}_{TH} T F \\ \frac{ⅆ T H}{ⅆ t} = T F ( 1 + {k}_{THn} T H ) - {k}_{TH} T F & ( {13}^{′} ) \\ \frac{D P C}{D t} = {k}_{PCTH} T H - {k}_{PC} PC + {S}_{PC} ( t ) & ( {14}^{′} ) \\ \frac{D B P}{D t} = {k}_{BP} ( 1 - B P ) - {k}_{{BPO}_{2}} {O}_{2} {f}_{s} ( N O ) + {k}_{{BPC}_{p}} {C}_{p} + {k}_{BPTH} T H ) B P & ( {15}^{′} ) \\ \frac{D D}{D t} = {k}_{DBP} ( 1 - B P ) + {k}_{DCp} {C}_{p} + {k}_{DO2} {O}_{2} + {k}_{DNO} N O / ( 1 + N O ) + {k}_{DEq} g ( {O}_{2} , N O ) - {k}_{D} D & ( {16}^{′} )\end{matrix}$$
so as to impute damage function calculations from the human data into the animal data and to enhance prediction of efficacy of said drug candidate.
